(19) United States
(12) Patent Application Publication
(10) Pub. No.: US 2026/0077150 A1
(43) Pub. Date: Mar. 19, 2026

(54) VENTILATOR AND METHOD FOR CONTROLLING A VENTILATOR

(71) Applicants: Ben FABRY, Erlangen (DE); Navid BONAKDAR, Erlangen (DE); Christian KUSTER, Effeltrich (DE)

(72) Inventors: Ben FABRY, Erlangen (DE); Navid BONAKDAR, Erlangen (DE); Christian KUSTER, Effeltrich (DE)

(73) Assignee: Ben FABRY, Erlangen (DE)

(21) Appl. No.: 19/106,065

(22) PCT Filed: Sep. 5, 2023
(86) PCT No.: PCT/EP2023/074318

§ 371 Date: Feb. 24, 2025

(30) Foreign Application Priority Data
Sep. 7, 2022 (DE) 10 2022 209 322.4

(51) Int. Cl. A61M 16/20; A61M 16/00; A61M 16/04; A61M 16/08; A61M 16/10

(52) U.S. Cl. CPC A61M 16/202; A61M 16/047; A61M 16/0833; A61M 16/1055; A61M 2016/0027; A61M 2016/0036; A61M 2230/40

(57) ABSTRACT

The invention describes a method device (1) for providing ventilatory assistance to a patient, comprising a set of tubes (30, 40, 50, 60, 70) for gas flow (fl) to and from the patient, a demand-flow valve (81, 81′), a flow sensor (86), and a pressure sensor (87, 88) for measuring the airway pressure. That demand-flow valve (81, 81′) is located at a Y-piece (8Y) that connects a tube (30) for the inspiratory and a tube (40) for the expiratory flow circuit to the patient. The invention further describes a method a demand-flow valve (81, 81′) which can be used for such a device (1) and to a method for controlling such a device (1).

The invention pertains a device for providing ventilator assistance to a patient, which device comprises a set of tubes for gas flow to and from the patient, a flow sensor, and a pressure sensor for measuring the airway pressure. Such a device can generally and also in the following be referred to as a “ventilator device” or “mechanical ventilator” or short “ventilator”. Therefore, these terms may be used interchangeably. Further, the invention pertains a demand-flow valve, which can be used for such a ventilator, and it pertains a method for controlling such a ventilator. In particular, the invention serves to provide ventilatory assistance to spontaneously breathing intensive care patients, especially those with respiratory insufficiency (generally and in the following also named “RI”) or acute respiratory distress syndrome (generally and in the following also named “ARDS”) in danger of baro- and volutrauma, additional work of breathing, and patient-ventilator-desynchronization.

For the sake of clarity, when referring to “airway pressure” in the context of mechanical ventilation and also in the context of the invention, it is generally measured outside the patient, within the ventilator circuit, at the proximal end of the “patient interface”, which connects the ventilator to the patient (e.g. this may be an endotracheal tube, generally and in the following also named “ETT”, a tracheostomy tube, generally and in the following also named “TT”, a face mask, or a breathing helmet). This pressure is also called “airway opening pressure” because it is measured near the point where the patient's airways are opening to the environment (note that the proximal end of the patient interface is also the patient's airways opening to the ventilator). By contrast, when referring to the biological airways in the patient's lungs, they will be referred to as “patient's airways”.

Intensive care patients that require ventilator support are typically intubated, sedated, if needed paralyzed, and ventilated using a form of pressure or volume-controlled ventilation. If the patient suffers from RI, e.g. due to pneumonia, or ARDS, e.g. due to sepsis, their lungs often become stiff during the course of their diseases, e.g. as a consequence of damage to the lung surfactant lining and the resulting increased air-liquid interfacial tension. Inflating the lungs with typical tidal volumes, e.g. 500-800 ml, then requires excessive inflation pressure. In addition, the lungs of RI and ARDS patients are often inhomogeneously ventilated, with parts of the lungs being underventilated, and others being overextended. Regional overextension of the lungs together with high pressure delivered by the ventilator can lead to baro- and volutrauma, leading to further lung damage and exacerbation of the disease. In addition, with increasing intra-thoracic pressure, the pulmonary vascular resistance increases and the return of venous blood to the heart decreases, leading to cardiac stress and reduced perfusion of other organs such as the brain and the kidneys.

To reduce the risk of baro- and volutrauma, intensive care physicians limit the maximum airway pressure during inspiration to below 30-35 mbar, and the tidal volume to about 6 ml/kg body weight. This single protective measure, when it was introduced into clinical practice by the late 1980s/early 1990, lead to dramatic decrease in the mortality of intensive care patients, from around 40-60% in the 1980 s, to 20-40% in the 1990s. This impressive success raised the hope that the mortality of RI and ARDS patients could be further reduced by an even more aggressive reduction of airway pressure and tidal volume. This, however, proved difficult, as the lungs of ARDS patients need a positive end-expiratory pressure (generally and in the following also named “PEEP”) of typically 10 mbar to prevent collapse, and with further reduced inspiratory pressure or tidal volume, the minute ventilation becomes too small for sufficient gas exchange. Therefore, it was suggested that RI and ARDS patients breath spontaneously, and the ventilator then augments each breath with some form of increased pressure support of a magnitude that is as low as possible but as high as necessary.

State of the art intensive care ventilators provide ventilatory modes that are intended to increase the airway pressure during the patient's inspiratory effort. The strategy is to provide only as much pressure support as is needed to maintain sufficient gas exchange without causing exhaustion of the patient. Such modes of patient-triggered inspiratory support have been proven clinically advantageous in ventilated patients, e.g. during weaning from mechanical ventilation, but thus far, these pressure support modes have largely failed in the case of RI and ARDS patients.

The reasons for this can be attributed to the following:

First, ARI and ARDS patients often show a rapid shallow breathing pattern. This makes it difficult for the ventilator to recognize inspiratory and expiratory efforts by the patient, especially if the pressure support is larger than the inspiratory pressure generated by the patient so that the patient's intrinsic breathing pattern is masked by that of the ventilator.

Second, the patient is typically intubated with an endotracheal tube (generally and in the following also named “ETT). The large and highly non-linear gas flow resistance of the ETT (due to turbulent flow conditions) dissipates large parts of the pressure support, which therefore never reaches the patient (Guttmann, Eberhard et al. 1993; all citations refer to the reference list at the end of the description, whereby the citations are characterised in the usual way by the indication of the first two authors and a year, if necessary by the addition of a number a or b, if several publications have been made by the same authors in the same year). Moreover, the ETT resistance hinders the expiration and causes an intrinsic PEEP to build-up in the patient's lungs (Guttmann, Eberhard et al. 1995). The ventilator, however, only measures the airway pressure outside the ETT (and, if applicable, the bacterial filter/humidified, which further adds to the airflow resistance). This further masks the patient's inspiratory and expiratory efforts.

The consequences of the ETT (and bacterial filter/humidifier) resistance, in combination with inadequate means of detecting the patient's inspiratory and expiratory efforts are severe:

Even though, when judged based on the time course of airway pressure, the ventilator seems to support the patient's spontaneous breathing pattern just fine, the patient's intratracheal and alveolar pressure are below PEEP during most of the inspiration, and far above PEEP during expiration. Thus, the patient must generate additional work of breathing and has great difficulty to exhale (Fabry, Haberthur et al. 1997).

Even more severe is the fact that the ventilator no longer recognizes the inspiratory and expiratory efforts of the patient. This may lead to an asynchrony between the patient's and the ventilator's breathing pattern, especially for patients with rapid, shallow breathing (Fabry, Guttmann et al. 1995). Patient-ventilator desynchronization means that about half of the time, the patients receive pressure support during their expiratory phases, which in effect increases the pressure load across the lungs instead of decreasing it, which was the original intension. In addition, about half of the time, the pressure support is reduced to PEEP-levels during the patients' inspiratory phases, which together with the large ETT resistance leaves the patient's breathing effort not only un-supported but causes substantial addition work of breathing.

Both problems—additional work of breathing and patient-ventilator desynchronization, can be, in principle, prevented by the ventilator mode Automatic Tube Compensation (generally and in the following also named “ATC”) (Fabry, Guttmann et al. 1994 a,b, Fabry, Haberthur et al. 1997). ATC controls not the airway pressure to a constant value (as in the ventilator mode continuous positive airway pressure (CPAP)), but instead the tracheal pressure.

This is done by continuously calculating the tracheal pressure (from the difference between airway pressure and pressure drop across the ETT tube, which in turn can be calculated from the product of the measured gas flow and the known ETT resistance) and controlling the gas flow to keep the tracheal pressure constant. Hence, the airway pressure is no longer set to a defined value during inspiration or expiration, but automatically changes to values larger than PEEP during inspiration, and values smaller than PEEP during expiration. The magnitude of this pressure swing depends on the breathing effort of the patient. ATC almost completely eliminates any additional work of breathing even for patients with high ventilator demand (Fabry, Haberthur et al. 1997), and completely prevents patient-ventilator desynchronization (Fabry, Guttmann et al. 1994 b).

To support ARDS patients with this mode, ATC can be combined with a flow- and/or volume-proportional assist ventilation (generally and in the following also named “PAV”). In essence, not the tracheal pressure, but either the alveolar pressure is continuously calculated and regulated (this corresponds to a flow proportional assist, which overcomes also the airway resistance), or the pleural pressure is continuously calculated and regulated (this corresponds to a volume proportional assist, which overcomes also the lung compliance). It is even possible to calculate and regulate the diaphragmatic muscle pressure (which corresponds to a flow- and volume proportional assist that overcomes ETT and airway resistance as well as lung and thorax compliance). Put differently, ATC with flow- and volume assist works similar to an E-bike where the bicyclist can choose the percentage by which his or her leg muscle efforts are being supported by an electric motor.

ATC with PAV was first implemented in an experimental ventilator ((Fabry, Guttmann et al. 1994 a), and after it was shown that RI and ARDS patients can be successfully ventilated with this mode (Fabry, Haberthur et al. 1997), it was also implemented in commercial ventilators. Today, ATC with PAV is a standard mode in intensive care ventilators. However, this mode is rarely used to support RI and ARDS patients. The reasons are unknown, but they may be a combination of several factors:

First, the rapid shallow breathing pattern of RI and ARDS patients (with tidal volumes below 300 ml and frequencies around 50-60/min) is not noticeable under standard pressure support (due to desynchronization), and only becomes apparent when the patient is ventilated with ATC (Fabry, Guttmann et al. 1994 b, Fabry, Guttmann et al. 1995). If clinicians are unfamiliar with such extreme breathing pattern, they may regard it as a sign of respiratory failure and switch to a mode that masks this pattern (the patient will still breath rapid and shallow, but the physician no longer sees it).

Second, commercial ventilators sense the airway pressure and gas flow typically within the ventilator, which stands about 2 m away from the patient. Hence, the speed of sound and the low-pass characteristics of the elastic tubing limit the reaction time of the ventilator. There is also the delay for the demand flow, which is generated in the ventilator 2 m away from the patient, and also cannot instantaneously reach the patient.

Third, the expiratory valve of commercial ventilators usually opens to atmosphere. Hence, the airway pressure is always positive. If the pressure difference across the expiratory valve, plus the expiratory tubing, plus the bacterial filter/humidifier, and plus the endotracheal tube, exceeds the PEEP-level, the tube resistance cannot be adequately compensated, hence ATC is no longer effective, and the patient's expiratory flow is reduced.

Overall, current solutions fail to provide ARDS patients with ventilator assist modes that allow them to breath spontaneously without risk of desynchronization, additional work of breathing, and consequently exacerbation of the underlying disease. Therefore, ARDS patients are typically ventilated with controlled modes and are not breathing spontaneously.

It is therefore an object of the invention to provide an improved device and corresponding method with which the aforementioned problems can be at least partially overcome or reduced.

This object is achieved by a device according to claim 1, a demand-flow valve according to claim 12, and a method according to claim 13.

A device according to the invention for providing ventilatory assistance to a patient comprises at least:
a set of tubes for gas flow to and from the patient,
a demand-flow valve
a flow sensor for measuring a gas flow in an airway to and from the patient, e.g. at the airway opening, and
a pressure sensor for measuring the airway pressure.

According to the invention, the demand-flow valve is located in the airflow circuit at a Y-piece that connects a tube for the inspiratory gas flow to the patient and a tube for the expiratory gas flow from the patient to a patient interface.

In general, a demand-flow valve is a pressure reducing device that regulates an outlet pressure to a set point regardless of the inlet pressure (which must be greater than the set point) and regardless of the gas flow. To accomplish this function, the valve resistance must be variable. A two-state (open-close) switching valve alone cannot function as a demand flow valve.

With such a demand-flow valve, the pressure which is generated at the patient's side of the set of tubes, and thus the gas flow to and from the patient, can be regulated or controlled (in the following, “control” and “regulation” are used synonymously, if not mentioned otherwise) directly, using suitable means for controlling the demand-flow valve, in particular on the basis of values measured by the flow sensor and pressure sensor. In contrast, with a switching valve, it is only possible to switch between the tube for the inspiratory and the tube for the expiratory gas flow circuit. In particular, the demand-flow valve, as explained later, may be used to control at least one of airway pressure, tracheal, alveolar, pleura, or muscle pressure, preferably depending on a mode of ventilator support.

As explained later, the Y-piece should be arranged as close as possible to the patient. When the demand-flow valve is located at the Y-piece according to the invention, it is also located in the airflow circuit as close as possible to the patient.

A demand-flow valve according to the invention is realized to be located at a Y-piece in the airflow circuit or gas flow tube system of a ventilator device. Some realization possibilities will be explained later.

The ventilator device according to the invention is preferably an intensive care device but may be also used in other scenarios for providing ventilator assistance to a patient.

A method for controlling a ventilator, in particular an intensive care ventilator, according to the invention comprises at least the following the steps:
measuring a gas flow in an airway to and from the patient, e.g. at the airway opening, with a flow sensor,
measuring an airway pressure in an airway to and from the patient, e.g. at the airway opening, with a pressure sensor,
controlling a demand-flow valve based on the gas flow and the airway pressure, whereby the demand-flow valve is located (close to the patient) at a Y-piece that connects a tube for the inspiratory and a tube for the expiratory flow circuit to the patient

The capability of direct control or regulation of the pressure from inspiratory flow to expiratory flow and back, in a demand-flow valve that is as close as possible to the patient, reduces at least the latency times between the point of control at which the pressure is changed according to a control signal and the point at which the current pressure change actually becomes relevant for the patient, namely at the exit of the patient interface or even deeper in the patient's airway (e.g. in the trachea or the alveoli). This allows for a more accurate demand-flow and airway pressure control and in principle, as will be explained later, also a performance of ventilatory modes as mentioned above, such as ATC and optionally PAV, that is (if used) improved regarding e.g. lower additional work of breathing, better synchrony with the patient's breathing efforts, lower intrinsic PEEP, and lower peak alveolar pressure levels.

Further, particularly advantageous embodiments and further embodiments of the invention are given by the dependent claims, as revealed in the following description. Features of different claim categories, description parts for different claim categories and individual features or feature groups of different embodiments may be combined as appropriate to give further embodiments.

As mentioned above, the Y-piece in a device or system for the supply of breathing air is an element that defines a branch point where the gas that is flowing to and from the patient's lungs and patient's airways is separated into the inspiratory and expiratory gas flow branches. The gas volume between the center of the Y-piece and the proximal end of the patient interface defines the functional dead space of the gas flow circuit. At the end of each expiration, the dead space is filled with oxygen-depleted alveolar gas, which is then re-inhaled at the beginning of the following inspiration. Therefore, it is preferred that this dead space is kept as small as possible. Consequently, the Y-piece should preferably be positioned in “close proximity” to the patient, and the distance of the demand-flow valve's patient-side outlet to the opening of the patient interface (which is the coupling point to the interface) should be accordingly small. This distance (which also may be used to define “in close proximity” or “close” to the patient) can range from 0 cm of the up to a maximum distance at which the maximally allowable dead space as specified by the attending intensivist is not exceeded. If for example the maximally allowable dead space for an adult patient with a body weight of 66 kg is 100 ml, the maximum distance is approx. 60 cm, depending on the diameter of the flexible tubing that connects the Y-piece with the patient interface, but a more preferable distance is max. 30 cm or even lower.

“Dead space” in the context used here is the volume of gas in the valve and connected tubing that contains previously exhaled air and is re-inhaled during the next breath, potentially affecting the patient's ventilation efficiency. In practice, a compromise must be found between reaction time and dead space on the one hand, which both decrease for shorter distances, and comfort for the patient and staff on the other hand, which increases with longer distances.

Preferably, the demand flow valve is implemented and positioned at the Y-piece such that the dead space in the demand flow valve and in a tubing system from the demand flow valve to a coupling point of a patient interface is below a value of 1.5 cm3 per kg body weight of the patient, or below a maximally allowable dead space as defined by the attending physician if a larger or smaller dead space is clinically warranted.

Preferably for adult patients, the demand-flow valve at the Y-piece is realized and positioned such that a maximum dead space in the demand-flow valve and in a tube system from the demand-flow valve to a coupling point of a patient interface is approx. 100 cm³, preferably 60 cm³, more preferably 55 cm3, further more preferably 30 cm3.

Preferably, the two tubes for the inspiratory and expiratory flow circuit that connect to the Y-piece are connected to a pressure source delivering a positive pressure for the inspiratory circuit and a negative pressure for the expiratory circuit.

The pressure source may be also part of the ventilator device, and may be realized by any arrangement of pressure sources for each tube or pressure side, e.g. blowers, preferably brushless radial blowers.

In one preferred embodiment, the pressure sources may be constant pressure sources delivering a near-constant positive pressure, preferably around +40 mbar, for the inspiratory circuit, and a near-constant negative pressure, preferably −20 mbar, for the expiratory circuit, whereby also other pressure levels can be chosen if needed, including atmospheric pressure, in particular for the expiratory circuit. In another preferred embodiment, the pressure sources, or at least one of the pressure sources, may also be variable, so long as the frequency and amplitude of pressure changes do not overwhelm the ability of the demand flow valve to control the flow and pressure at the patient side of the valve to a desired value.

In particular, the pressure sources can also preferably be controlled (coordinated with the control of the demand-flow valve) in such a way that they are raised and lowered as needed, for example, that only if a certain pressure is needed at all, the corresponding pressure source also generates a corresponding pressure and otherwise the pressure source only generates less pressure or is even switched off. This can be advantageous to make the device quieter. For this purpose, suitable pressure sources (e.g. blowers) can be used that can be switched accordingly quickly. It should be noted that, as usual in the field of ventilation technology, all pressures values given here are the pressure difference to the current ambient pressure (nominally the current atmospheric pressure on site) and, therefore, common differential pressure sensors are used (unless otherwise stated).

In principle, the demand-flow valve at the Y-piece may be realised in various ways. For example, controllable closing elements or shutters (e.g. flaps), can be arranged at or in the tubes or tube connections for the inspiratory and the expiratory flow circuit directly neighboured to the Y-piece (and optionally also at or in the tube or a tube connection to the patient).

Preferably, the demand-flow valve is realized such that the pressure and flow at the inspiratory side can be regulated while the expiratory side remains closed independent of the pressure at the inspiratory side, or vice versa. On the one hand, during the inspiration phase, the expiratory flow circuit can remain closed by the demand-flow valve while at the same time flow and pressure of the inspiratory flow circuit can be regulated by the demand-flow valve. On the other hand, during the expiratory phase, the inspiratory flow circuit can remain closed by the demand-flow valve, while at the same time the flow and pressure of the expiratory flow circuit can be regulated by the demand-flow valve. Such a design can save a considerable amount of breathing gas and reduce the O2 consumption.

Preferably, the Y-piece houses the demand-flow valve, or, in other words, the demand-flow valve may be integrated in the Y-piece, or, in other words, the demand-flow valve functions as the Y-piece. In this way, the valve can be kept as compact as possible, and the regulation or controlling of the pressure and the gas flow to and from the patient can be realized as directly as possible at the point where the inspiratory circuit and expiratory flow circuit meet.

However, preferably the demand-flow valve may be a preferably rotating or better rotatable 3-way valve (for example, a valve with a rotatable closure element (which may also be referred to as a shutter) inside a valve housing, as described later in detail), which connects the outlet (towards the patient) either to the positive or negative side. Or, in other words, which connects the outlet towards patient interface with the inspiratory side (which is normally under positive pressure) to the expiratory side (which is normally connected to negative pressure or the atmosphere). More preferably, the 3-way valve may be a rotating 3-way valve.

The use of a 3-way valve, and in particular a rotating or rotatable 3-way valve, allows for a particularly simple and compact design and particularly easy and exact control of the pressure and gas flow.

Further, such a 3-way valve allows also an easy realization of a demand-flow valve such that the pressure and flow at the inspiratory side may be regulated while the expiratory side remains closed or vice versa, as already mentioned above.

The pressure inlets (the connections to the gas streams of inspiratory gas, usually on positive pressure, and of expiratory gas, preferably on negative pressure) may preferably connect to the valve housing either in a straight or curved arrangement; the outlet can be either in-plane with the inlet, or preferably perpendicular to the inlets, or otherwise tilted. Especially preferred embodiments for both versions will be described later.

Curved pressure inlets in combination with a perpendicular outlet may allow for a more compact design and may further reduce the inner volume of the valve (and the dead space).

Preferably, means to prevent flow turbulence, e.g. a grid structure, an array of laminar tubes or the like, may be positioned in the outlet of the demand-flow valve.

Preferably, the flow sensor and the pressure sensor for measuring the airway pressure (or airway opening pressure), which may be used to as a basis for controlling the demand-flow valve, are also located in close proximity to the patient as defined above. This may help to reduce the delay of the feedback control signals for the demand-flow valve. Further preferably, the flow sensor and the pressure sensor are placed on the patient's side (the side which leads to the patient interface) of the demand-flow valve. If the airway pressure is probed close to the patient downstream (proximal) from the demand-flow valve, this automatically ensures that all upstream (distal) resistive elements are fully compensated, such as the resistance of the tubing and the demand-flow valve.

In an especially preferred configuration, the Y-piece, which preferably houses the demand-flow valve, connects (on the patient's side) to a pneumotachograph or other device for flow sensing. This in turn optionally connects to a bacterial filter and/or humidifier, which is preferably followed by a short flexible tube that connects to a swivel connector and further to a patient interface (e.g. an ETT, TT, face mask or breathing helmet).

However, instead of using a combination of bacterial filter and/or humidifier, especially for long-term ventilated patients, an active humidifier can also be located in the inspiration circuit (inspiration tube) to the Y-piece, which may better humidify the inspiration air.

As already mentioned above, in order to control or regulate the supplied pressure or gas flow to and from the patient, the ventilator may comprise a means, respectively a controller (e.g. a controlling unit or system), for controlling or regulating the demand-flow valve. Accordingly, this controller is connected to the demand-flow valve and, preferably, as the control or regulation should be done (directly or indirectly, as explained below) on the basis of the values measured by the flow sensor and pressure sensor, is also connected (directly or indirectly) to the flow sensor and pressure sensor.

The controller comprises preferably a PID controller (proportional-integral-derivative controller), but other control algorithms can be used.

Advantageously, the control or regulation of the pressure, which is provided at the patient's side, and of the gas flow to and from the patient, may depend on a predefined (or pre-set) mode of ventilator support. Preferably, the controller or controlling system of the ventilator can operate in a predefined mode, which may—via a user interface—configured by a user or selected by a user, from a number of pre-set modes, which may be stored in the controlling system, for example.

Preferably, the invention may be used to control (respectively regulate) at least one of an airway pressure, a tracheal pressure, an alveolar pressure, a pleura pressure or a muscle pressure, or fractions thereof, by controlling the demand-flow valve. In particular, in the predefined mode of ventilator support it may be specified with regard to which pressure (airway pressure, a tracheal pressure, an alveolar pressure, a pleura pressure or a muscle pressure) the control method may be optimised.

For example, the target airway pressure can be set according to established ventilatory modes, such as patient-triggered inspiratory pressure support. The advantages of the proposed demand-flow method and device, however, are most apparent in ventilatory modes that require a fast and precise response of the demand-flow valve to a rapidly changing demand, in particular, in the mode ATC without or with proportional assist ventilation (ATC+PAV). The mode ATC+PAV compensates downstream resistive and elastic pressure differences between the demand-flow valve and the patient's respiratory muscles, and therefore reduces or avoids excessive work-of-breathing, excessive negative of positive transpulmonary pressure, intrinsic PEEP, or patient-ventilator desynchronization.

The actual tracheal pressure, alveolar pressure, pleura pressure or muscle pressure may be, preferably continuously, calculated based on the gas flow and the airway opening pressure. For example, the tracheal pressure may be calculated from the difference between airway pressure and pressure drop across the ETT tube, which in turn can be calculated from the product of the measured gas flow and the known ETT resistance.

When the ventilator is operated in the ATC+PAV mode, the target pressure P_{target} of the controller may be compute as follows:

$${P}_{target} = PEEP + {f}_{0} \cdot Δ P ( ETT ) + {f}_{1} \cdot Δ P ( {R}_{aw} ) + {f}_{2} \cdot Δ P ( {E}_{rs} ) \qquad ( 1 )$$

where PEEP is the positive end-expiratory pressure, ΔP(ETT) is the pressure drop across the endotracheal tube and any additional resistive elements such as a bacterial filter/humidifier, swivel connector, CO2 sensor, bronchoscope or catheter inserted into the tube; ΔP(R_{aw}) is the pressure drop caused by the airway resistance; and ΔP(Eᵣₛ) is the pressure drop caused by the elastance of the respiratory system. The factors f₀, f₁ and f₂ are adjusted by the attending intensivist or respiratory therapist to be between zero and unity, whereby a value of unity results in full (100%) compensation of the work of breathing caused by the respective resistive or elastic element, and a value of zero results in no (0%) compensation.

These pressure drops can be computed based on the continuously (e.g. with the pneumotachograph) measured gas flow V′ and volume V, which is the time integral of the gas flow (V=∫V′ dt, with boundary condition V=0 at the beginning of inspiration) as follows:

The pressure drop ΔP(ETT) during inspiration (V′ is positive) can be calculated according to

$$Δ P ( ETT ) = {k}_{1 , in} \cdot {\dot{V}}^{{k}_{2 , in}} \qquad ( 2 a )$$

$$or alternatively$$

$$Δ P ( ETT ) = {k}_{1 , in} \cdot \dot{V} + {k}_{2 , in} \cdot {\dot{V}}^{2} \qquad ( 2 b )$$

and during expiration (V′ is negative)

$$Δ P ( ETT ) = - {k}_{1 , ex} \cdot {( - \dot{V} )}^{{k}_{2 , ex}} \qquad ( 3 a )$$

or alternatively

$$Δ P ( ETT ) = {k}_{1 , ex} \cdot \dot{V} - {k}_{2 , ex} \cdot {\dot{V}}^{2} \qquad ( 3 b )$$

In Equations (2a, 2b, 3a, 3b), the coefficients k_{1.in}, k_{2.in}, k_{1.ex}, k_{2.ex} describe the non-linear resistance of the endotracheal tube and additional resistive elements, as described in (Guttmann, Eberhard et al. 1993). Other empirical fit equations, apart from a power-law fit (Eq. 2a, 3a) or a second-order polynomial (Eq. 2b, 3b) are also possible.

The pressure drop ΔP(R_{aw}) across the patient's airways that is caused by the patient's airway resistance R_{aw} can be calculated according to

$$Δ P ( {R}_{aw} ) = {R}_{aw} \cdot \dot{V} \qquad ( 4 )$$

The pressure drop ΔP(Eᵣₛ) across the patient's lungs and relaxed chest wall (respiratory and abdominal muscles are relaxed) that is caused by the patient's elastance of the respiratory system Eᵣₛ can be calculated according to

$$Δ P ( {E}_{rs} ) = {E}_{rs} \cdot V \qquad ( 5 )$$

In Equation (1), factors f₀, f₁ and f₂ may be chosen between zero (0% compensation) and unity (100% compensation). f₁ and f₂ are preferably set to zero during expiration. Preferably, f₀ is chosen to be unity during both inspiration and expiration. This ensures that the tube resistance and other added resistive elements cause no additional work of breathing and no intrinsic PEEP. In some cases, e.g. in COPD patients that suffer from expiratory flow limitation, a f₀ value below unity may be chosen during expiration. Since the precise values of R_{aw} and Eᵣₛ are often not known, f₁ and f₂ may also be chosen to be less than unity, to avoid so-called run-away effects. Run-away effects can also be prevented by setting f₁ and f₂ to a lower value once a set tidal volume has been reached.

From these pressure drops, the tracheal pressure P_{trach}, alveolar pressure Pₐₗᵥ, or muscle pressure Pₘᵤₛ can be computed as follows:

$${P}_{trach} = {P}_{aw} - Δ P ( ETT ) \qquad ( 6 a )$$

$${P}_{alv} = {P}_{trach} - Δ P ( {R}_{aw} ) \qquad ( 6 b )$$

$${P}_{mus} = {P}_{alv} - Δ P ( {E}_{rs} ) \qquad ( 6 c )$$

If the actual tracheal pressure, alveolar pressure, pleura pressure and/or muscle pressure are known, the gas flow may be controlled, using the demand-flow valve, to keep the tracheal pressure constant and/or to compensate fully or partially for the work of breathing required to overcome the airway resistance, lung elastance, and chest wall elastance. In this way, the invention may help to minimize the additional work of breathing for the patient, caused by the resistance of the various elements in the inspiratory and expiratory circuit (in particular the ETT, the resistance of the humidifier, the resistance of the expiratory tubing and expiratory valve) and the delay of the demand-flow and the feedback control signals (airway pressure gas flow).

Furthermore, the controller may also be realized to control the positive and negative pressure sources, in particular to modify or modulate the pressure levels delivered by the positive and negative pressure sources. For example, if the pressure sources comprise brushless radial blowers, the exact pressure may easily be adjusted or controlled by changing the motor speed.

In one embodiment, the demand-flow valve is realized such that in a neutral position of the demand-flow valve, a small overlap allows flow to pass between the positive (inspiratory) and negative (expiratory tube) side. This may help to improve pressure control accuracy under low-flow conditions (e.g. at the end of expiration).

The “neutral position” of the demand-flow valve is a middle position of the valve, meaning in the middle between an inspiratory position, in which the demand-flow valve connects the tube for the inspiratory flow circuit to a tube to the patient, and an expiratory position, in which the demand-flow valve connects the tube for the expiratory flow circuit to a tube to the patient.

However, in another embodiment, the demand-flow valve has no overlap in the neutral position, because this reduces O₂ consumption and allows an occlusion manoeuvre, if desired.

Since both variants may be advantageous depending on the situation, the demand-flow valve can preferably also be realized such that it can be switched into a mode with overlap in the neutral position and into a mode without overlap in the neutral position.

A ventilator according to the invention may no longer (as the state of the art intensive care ventilators) be seen as a single device, which is usually located 1.5-2 m away from the patient, but is instead separated into two main parts. Preferably, the ventilator comprises:
A valve-sensor-assembly (in the following also called “valve-sensor-unit”), which is placed close to the patient at the location of the Y-piece, comprising the demand-flow valve and the sensors. Thereby, the demand-flow valve of this valve-sensor-unit may replace the Y-piece.
A base station, which is located remotely from the valve-sensor-assembly and typically 1.5-2 m away from the patient. This base station may comprise at least the means for generating (and controlling, if desired) the positive and negative inlet pressure.

Preferably, in this embodiment, the remote base station on the one hand
may provide the air/oxygen mixture from the air and oxygen pressure lines of the hospital (or from a compressed air and oxygen cylinder);
may generate the positive and negative pressure for the inlet and outlet e.g. with the help of a radial blower, Venturi valve, pressure regulator etc.,
may contain the power supplies for all parts of the device,
may optionally contain a computer or controller for various monitoring and control tasks and for providing a user interface (e.g. for selecting the mode of ventilation, device setup),
may optionally house a monitor for visual feedback and for displaying the measured flow, pressure and volume curves and other respiratory parameters.

The latter two optional components (computer and monitor) could also be provided e.g. by a separate laptop computer, tablet, smartphone, or other remote monitoring and control device.

On the other hand, preferably the valve-sensor-unit
may continuously measure the airway pressure and the gas flow
may contain a motorized valve that controls the airway pressure or gas flow to a desired value, depending on the selected ventilatory mode,
may optionally also contain a microcontroller for data acquisition, for controlling the valve, or for operating the ventilator.

Examples for preferred embodiments of a base station and a valve-sensor-unit will be explained later in more detail in the context with reference to the figures.

In most of the cases in which intensive care patients need ventilator assistance, an ETT (endotracheal tube) is used as the patient interface from the ventilator to the patient's airways. However, while it may seem tempting for the intensivist to select an endotracheal tube with a larger diameter to reduce its resistance and improve airflow, this can cause other serious complications. Immediate complications of a larger tube diameter include irritation, inflammation, trauma, and pressure ulcers of the delicate tracheal tissues, subglottic stenosis, and vocal cord damage. Long-term consequences of a large tube diameter after extubation can include impaired breathing, speaking and swallowing, severely affecting the patient's overall well-being and recovery.

As explained above, the invention allows a better use of methods like ATC (with or without PAV), which aim to compensate the resistance of the components in the gas flow circuit and in particular in the ETT. Therefore, in a preferred embodiment of the invention, the set of tubes for gas flow to and from the patient comprises, in a version for adults, at least one endotracheal tube or tracheostomy tube which has a maximum inner diameter of 7 mm, preferably 6 mm, more preferably 5 mm, and if feasible even smaller. In a version for child or infant patients, the set of tubes for gas flow to and from the patient comprises at least one endotracheal tube or tracheostomy tube which has a maximum inner diameter of 5 mm, preferably 2 mm.

In some situations, it is desirable or necessary to insert an additional tube-shaped medical instrument through the ETT or TT, such as an endoscope and especially a bronchoscope. This also significantly increases the resistance in the ETT or TT. However, as explained above, the invention gives the opportunity for a better compensation of any resistance in the tube. Therefore, in a preferred embodiment of the invention, the set of tubes for gas flow to and from the patient comprises at least on ETT or TT, whereby an instrument lead and/or tube (e.g. an bronchoscope, endoscope or other tube shaped instrument) is arranged inside the ETT or TT to extend along at least a portion of the length of the ETT or TT.

Overall, the invention provides a fast and precise demand-flow method and demand-flow valve (and a ventilator with such a demand-flow valve) for controlling airway pressure and gas flow for mechanically ventilated patients. Particularly advantageous features and effects are summarized again below:

A first feature is that the demand-flow valve is placed in close proximity to the patient; specifically, it may replace the traditional Y-piece that connects the two tubes for the inspiratory and expiratory flow circuit to the patient. The advantage of this close proximity to the patient's airway is to reduce the reaction time caused by the speed of sound, by the flexibility of the tubing, and by pressure oscillation in the air column.

To further reduce the reaction time of the demand flow valve, a second feature is that the flow and pressure sensors may also be placed in close proximity to the patient, specifically between the demand-flow valve and the ventilator-end of the patient interface (e.g. ETT, TT, face mask or helmet). This improves the performance and accuracy of the demand flow control as the speed of sound, the flexibility of the measurement tubing, and pressure oscillation in the air column in the tubing no longer cause appreciable delays in the delivery of flow and pressure.

Since the demand-flow valve is now in close proximity to the patient, it is as a third feature that the feeding (inlet) pressure of the valve can be low (compared to the pressure of several atmospheres of the oxygen and compressed air pressure lines that are typical for hospitals). This is advantageous for patient safety reasons. The pressure of the inlet does not need to be closely controlled, as the demand flow valve can deal with large pressure fluctuations. The pressure should preferably be adjusted to the lowest level sufficient to deliver the desired airway pressure and gas flow. For the majority of situations, a positive pressure of +40 mbar will be sufficient, but higher or lower values may be needed depending on the patient's breathing pattern and respiratory mechanics. For example, for ETTs with inner diameters below 5 mm, or during bronchoscopy where an endoscope is introduced into the endotracheal tube, the resulting tube resistance can exceed values of 100 mbar*s/l. In this case, a supply pressure greater than +100 mbar can be recommended.

A fourth feature is that a separate expiratory valve, also called PEEP (positive end-expiratory pressure) valve, which is an essential component of traditional ventilators, is no longer needed. This is because the demand flow valve is capable of regulation the airway pressure both during inspiration and expiration. The advantage of controlling expiratory pressure by the demand-flow valve is that the resistance of the expiratory tubing can be automatically compensated.

Optionally, as a fifth feature, the outlet of the demand-flow valve opens not to atmospheric pressure (as is customary in traditional ventilators) but is connected to a negative pressure source. This allows the demand flow valve to compensate for the flow resistance of the ETT during expiration and thus helps to reduce or avoid intrinsic PEEP, also called auto PEEP. For the majority of situations, a pressure of −20 mbar will be sufficient, but less or more negative values may be needed depending on the patient's breathing pattern and the resistance of the ETT, as explained above in connection with the third feature. As with the positive pressure of the inlet, the negative pressure of the outlet does not need to be precisely controlled as the demand-flow valve can effectively deal with pressure fluctuations.

A sixth feature is that a ventilator featuring the new demand flow valve no longer is a single device located 1.5-2 m away from the patient but now is separated into two parts, the valve-sensor-unit (demand-flow valve and sensor unit) close to the patient, and the remote base station, as already explained above.

In particular in combination, these features allow for more accurate and faster demand-flow control. The demand flow valve and method, combined with the automatic tube compensation control algorithm, allow for near complete compensation of tube resistance regardless of tube diameter. This eliminates the concern that a small tube will cause excessive resistance and compromise ventilation. Instead of a standard (inner) tube diameters of 8 mm for men and 7 mm for women, tube diameters down to 4.5 mm are feasible (for an inlet and outlet pressure of around 100 mbar; with larger inlet and outlet pressure levels, the resistance of even smaller tube diameters can be compensated). By analogy, a similar relative reduction in tube diameter is possible when the system is used for infants and children. Smaller tube diameters help to significantly reduce tracheal tissue damage, subglottic stenosis, and vocal cord damage, and thereby also reduce complications after extubation such as impaired breathing, speaking and swallowing.

Beyond compensating for the tube resistance, the proposed method and device for demand flow control can moreover be used to compensate for additional resistive elements such as a humidifier and bacterial filter, or a bronchoscope, catheter, or measurement probe that has been inserted through the endotracheal tube.

Other objects and features of the present invention will become apparent from the following detailed descriptions considered in conjunction with the accompanying drawings. It is to be understood, however, that the drawings are designed solely for the purposes of illustration and not as a definition of the limits of the invention. In the diagrams, like numbers refer to like objects throughout. Objects in the diagrams are not necessarily drawn to scale.

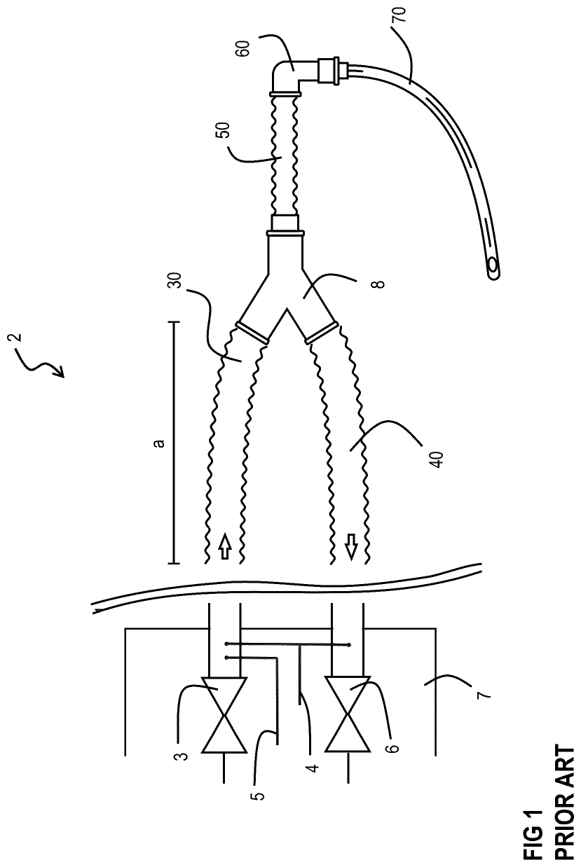

FIG. 1 a schematic representation of components of a state-of-the-art ventilator and their arrangement relative to each other;

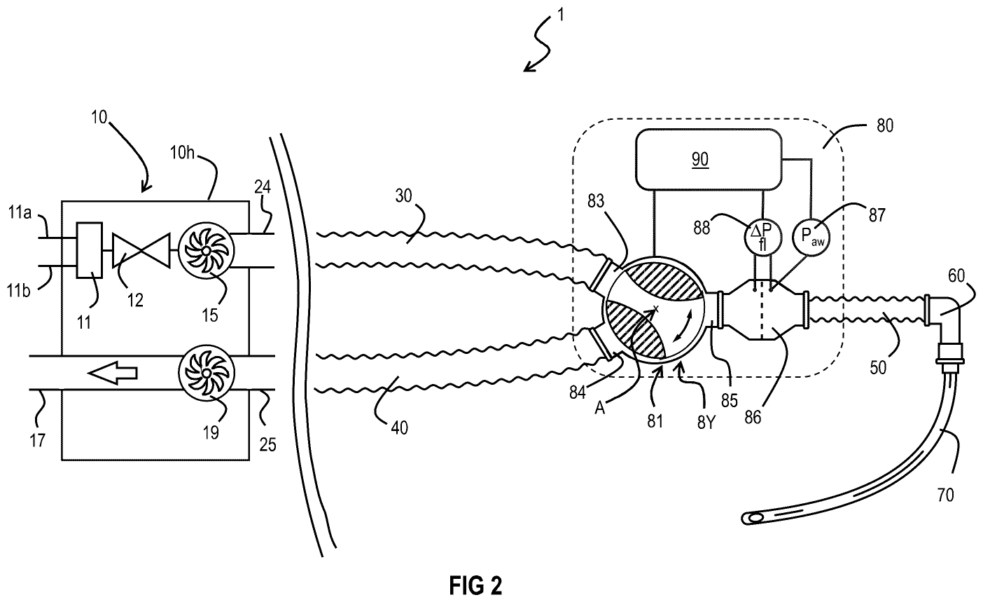

FIG. 2 a schematic representation of components of a first embodiment of a ventilator according to the invention and their arrangement relative to each other;

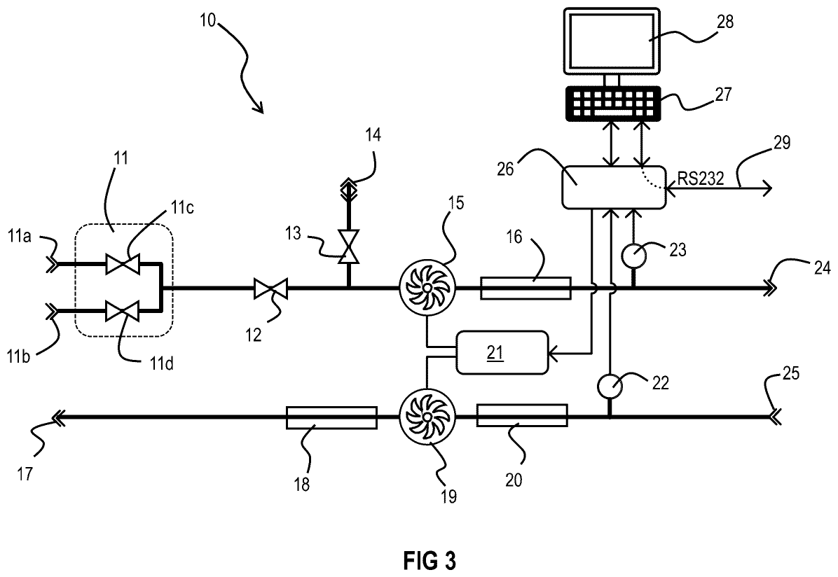

FIG. 3 a schematic representation of components of an embodiment of a base station for a ventilator according to the invention;

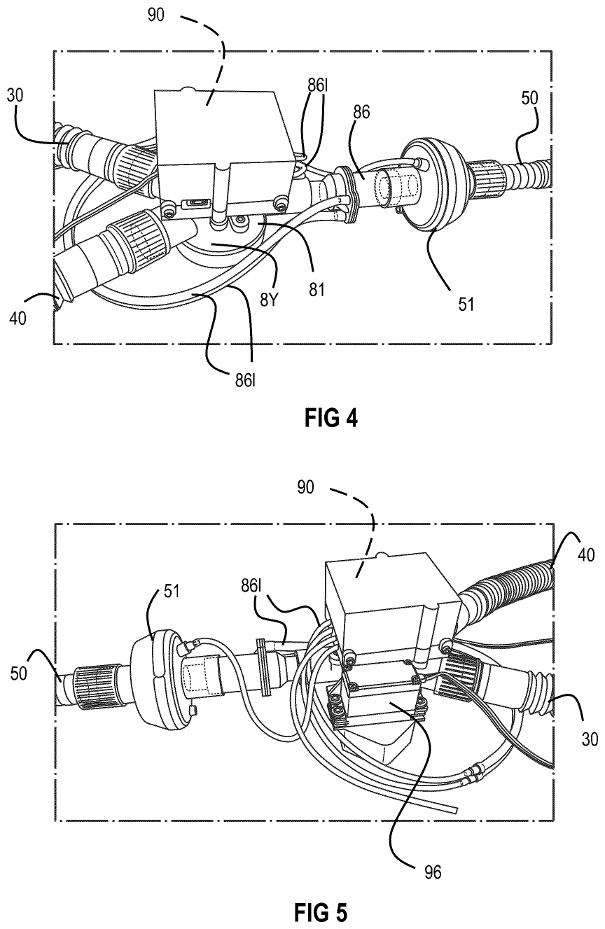

FIG. 4 a first schematic view of a valve-sensor-assembly for a ventilator according to the invention comprising a first embodiment of a demand-flow valve;

FIG. 5 a second schematic view of the valve-sensor-assembly of FIG. 4;

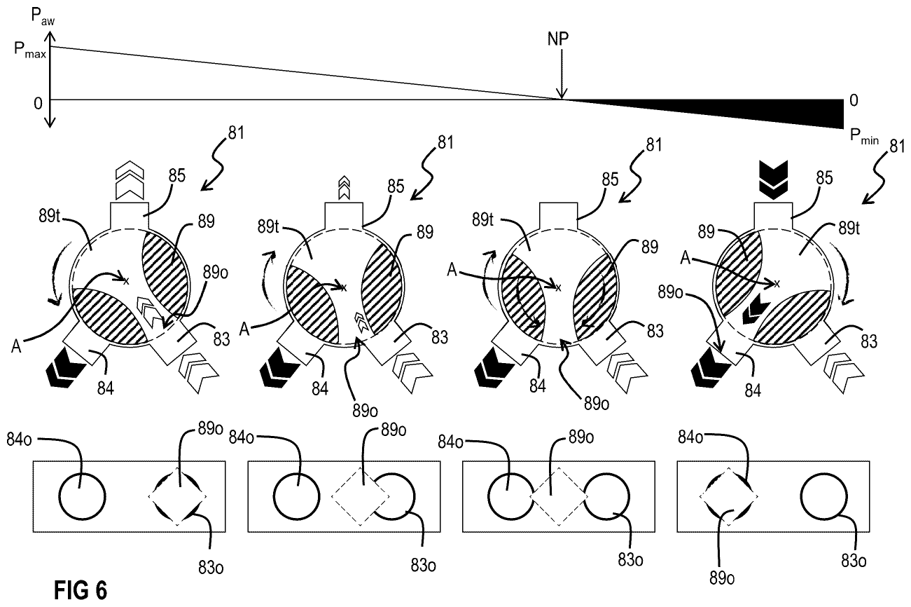

FIG. 6 a schematic representation of the operation of the demand-flow valve for the valve-sensor-assembly of FIGS. 2 and 4 in various rotational positions of the demand-flow valve;

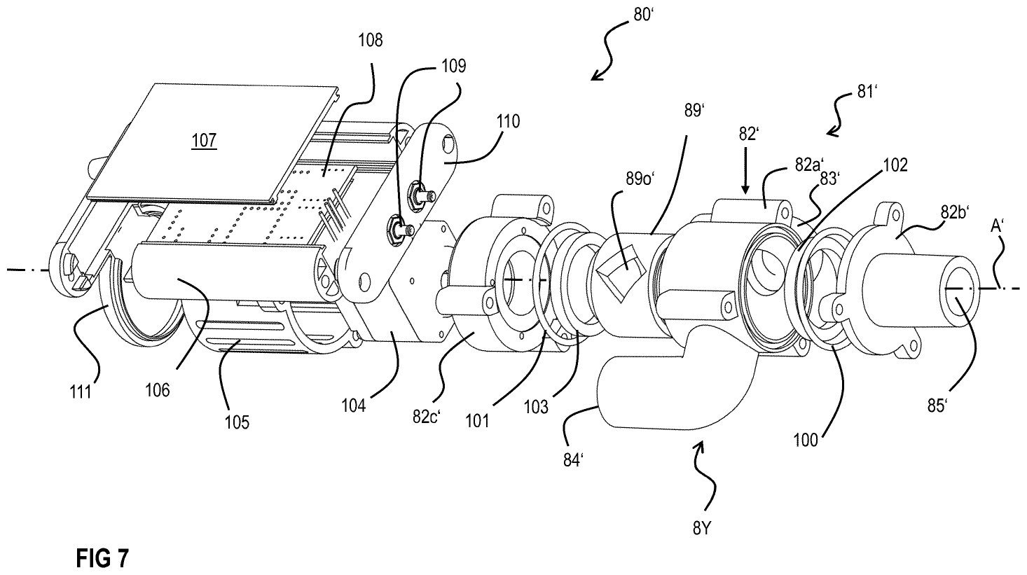

FIG. 7 a schematic exploded view of a second embodiment of a demand-flow valve for a valve-sensor-assembly for a ventilator according to the invention;

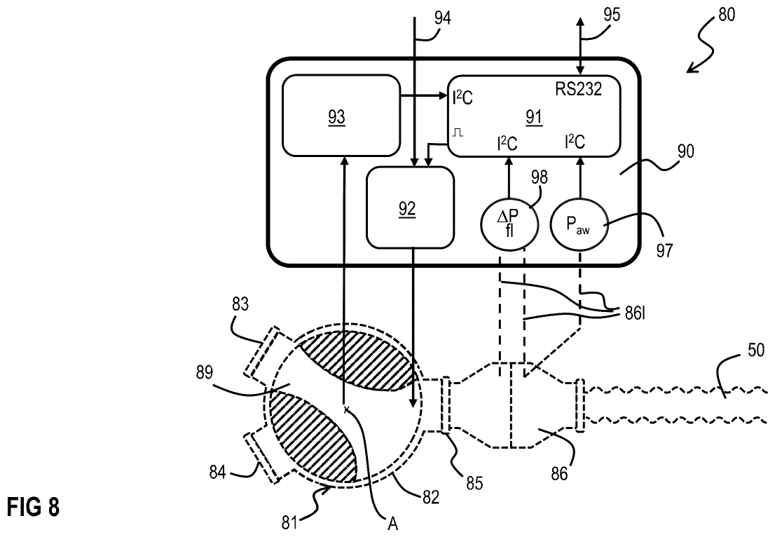

FIG. 8 a schematic representation of electronic components of an embodiment of a controller for a valve-sensor-assembly for a ventilator according to the invention;

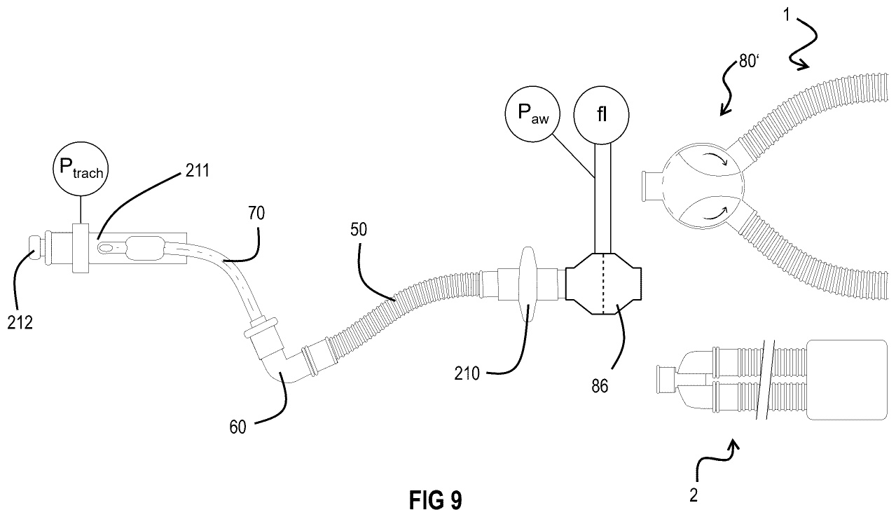

FIG. 9 a schematic representation of an experimental setup for testing and comparing the prototype ventilator with a commercial ventilator;

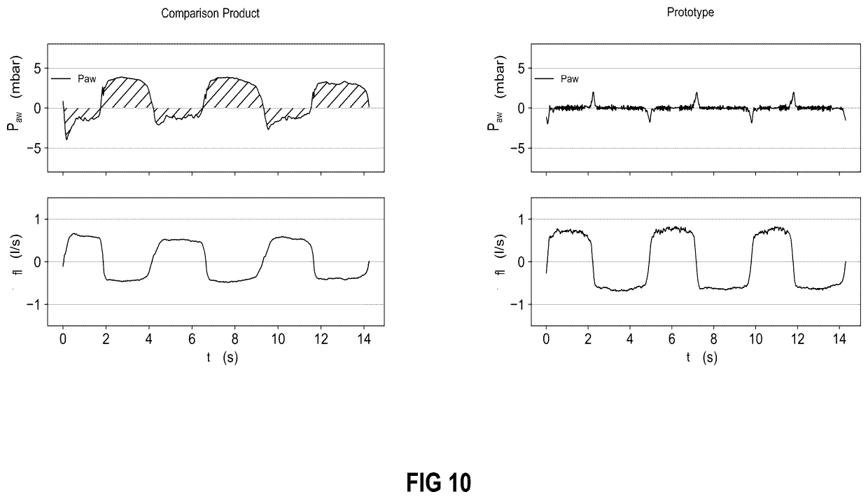

FIG. 10 different measurement diagrams of pressure and gas flow over time for a leading conventional ventilator (left diagrams) compared to a ventilator according to the invention (right diagrams) in a first operating mode (CPAP);

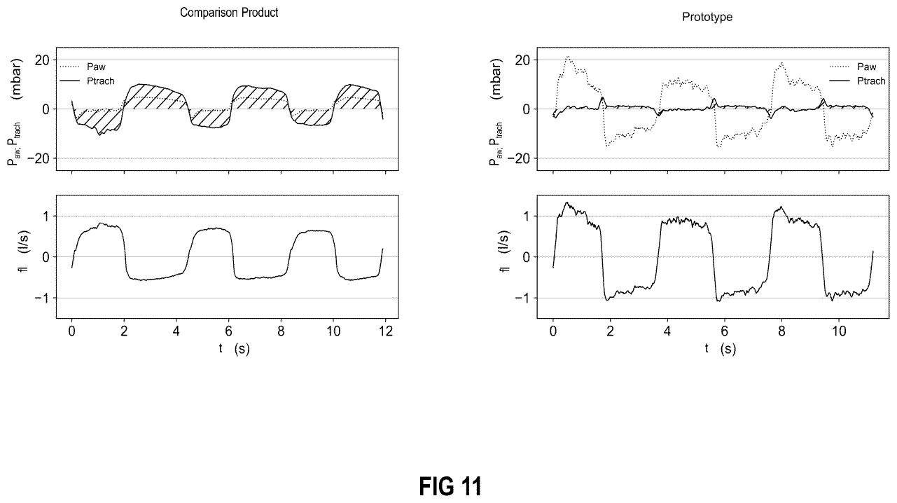

FIG. 11 different measurement diagrams of pressure and gas flow over time for a leading conventional ventilator (left diagrams) compared to a ventilator according to the invention (right diagrams) as in FIG. 10 but in a second operating mode (ATC).

FIG. 1 shows a schematic sketch of a typical setup of the connection of a state-of-the-art ventilator 2 with a patient (not shown). In most cases of mechanical ventilation, an ETT 70 is inserted into the patient's trachea. This ETT is connected to a flexible tube 50 via a swivel connector 60. The flexible tube 50 is connected at its distal end to the patient-side outlet of a so-called Y-piece 8. Here, the gas flow circuit is divided into two branches, namely a first tube 30, to supply inspiratory gas from a ventilation unit 7 to the patient during the inspiration phase, and a second tube 40, which leads to an outlet valve 6 in the ventilation unit 7 to let the exhausted gas out during the exhalation (=expiration) phase.

The pressure and the gas flow supplied to the patient during the inhalation phase is controlled via a demand-flow valve 3, which is located in the ventilation unit 7. Alternatively or additional to the demand-flow valve 3, a suitable blower (not shown) may be controlled directly to control pressure and gas flow. In addition, sensors 4, 5 for pressure and gas flow are located inside the ventilator unit 7 directly behind the valves 3, 6 in the direction of the patient. On the basis of the values measured by these sensors 4, 5, the valves 3, 6 may be controlled.

The Y-piece 8 is generally arranged at a distance a of approx. 1.5-2 m from the ventilation unit 7 in the tube system. Thus, the valves 3, 6, in particular the demand-flow valve 3, and the sensors 4, 5 are correspondingly far away from the patient, namely usually around 2 m. This positioning leads to the problems mentioned above.

For comparison with this prior art, FIG. 2 schematically shows the setup of an example of a ventilator device 1 according to the invention. Here, too, the ETT 70 is connected to a swivel connector 60, which in turn is connected to the flexible tube 50.

In contrast to the state of the art, however, the flexible tube 50 is not connected at its distal end to a conventional Y-piece, but rather to a valve-sensor-assembly 80, which comprises a controllable demand-flow valve 81 and measuring device 86, 87, 88 for measuring the pressure and the gas flow, here a pneumotachograph 86 (in which, as will be explained later, a pressure P_{aw} and a pressure difference ΔP are determined with the aid of two pressure sensors 87, 88 and thus the gas flow fl is determined as usual). Here, the demand-flow valve 81 has the function of a Y-piece, because in the demand-flow valve 81 the gas flow circuit is divided into the two branches, namely the first tube 30 (inspiratory tubing 30) for inspiratory gas during the inspiration phase, and a second tube 40 (expiratory tubing 40) to let the exhausted gas out during the exhalation phase. In other words, the demand-flow valve 81 is a kind of Y-piece 8Y with an integrated demand-flow valve and PEEP-valve function.

This valve-sensor-assembly 80 is located relatively close proximity to the patient, which results in a relatively small dead space from the distal end of the patient interface 70 (here the ETT 70) up to the demand-flow valve 81. In a typical configuration for adult patients as shown in FIG. 2, the demand-flow valve 81 may be connected to the patient via the pneumotachograph (length ˜5 cm), the flexible tube 50 (contour length ˜20 cm), and the swivel connector 60 (contour length ˜5 cm). Then, the total distance from the airway opening is 30 cm, and the total dead space is 51 cm³ (13 cm³ from the demand-flow valve prototype, 5 cm³ from the pneumotachograph, 29 cm³ from the flexible tube, and 4 cm³ from the swivel connector). The distal end of the patient interface 70, here the connection point from the ETT 70 to the swivel connector 60, is defined as the “airway opening”, as already explained above. If, optionally, a passive humidifier/filter is added between the pneumotachograph and the flexible tube, as for example shown in FIGS. 4 and 5, this will increase the dead space by another 5 cm³.

It should be noted, that the ETT 70 is only one example for a patient interface 70. Instead of an ETT 70, another patient interface, for example a TT, a face mask or a helmet or the like, can also be used, depending on the situation.

As also shown in FIG. 2, in upstream direction from the Y-piece 8Y with the integrated demand-flow valve 81, away from the patient, each tube 30, 40 is connected with a corresponding port 24, 25 in a housing 10h of a base station 10 of the ventilator 1.

The circuit for the inspiration branch leads inside the housing 10h to a first controllable blower 15 to which breathing air is supplied by an air-O₂-blender 11 via a pressure reducing valve (pressure regulator) 12. This blower 15 ensures a defined, adjustable overpressure, for example between 0 and approx. +100 mbar, above the ambient pressure to support inhalation.

The circuit for the exhalation branch leads to a second controllable blower 19, which is connected on the other side to an outlet 17 for the used air and which, if required, can supply on its suction side (side towards the patient) a negative pressure, for example between 0 and −100 mbar, relative to the ambient pressure in order to support exhalation.

Both the structure of the base station 10 and the structure of the valve-sensor-assembly 80, in particular the demand-flow valve 81, as well as their modes of operation will be explained in more detail below with reference to FIGS. 3 to 8, based on various embodiment examples.

First, the structure and functions of the base station 10 are explained in more detail using FIG. 3, which provides an overview of the arrangement of various components in the base station 10. Not shown here for clarity is the power supply (for the computer/microcontroller, blower motors and DC motor servo controller, and—if requested—for the demand flow valve and the valve controller in a valve-sensor-assembly, which is positioned close to the patient).

The oxygen mixture for setting a requested inspiratory oxygen fraction (Fi,O₂) is provided by an air-oxygen blender 11 fed via ports 11a, 11b from the hospital's central gas supply or from compressed gas cylinders. The blender 11 comprises pressure reducing valves 11c, 11d, to reduce the gas pressure to a typical value of ˜1.5 bar, which is then further reduced with a pressure regulator 12 to close to atmospheric pressure (0 mbar). A breathing valve 13 (a pressure/vacuum relief valve 13 that opens via a port 14 to atmosphere) ensures that the pressure at this point remains close to zero in case of a malfunctioning of the valves 11c, 11d or a blower 15, which will be explained in the following.

The air-oxygen mixture is then pressurized using a (radial) blower 15 to values up to +100 mbar (typically +40 mbar), which is then delivered to the demand-flow valve via the port 24 and the inspiratory (=inspiration) tubing 30. To reduce high frequency acoustic noise, a silencer 16 or muffler can be used.

It is also possible to generate the pressure directly with an electronically, pneumatically, or manually controlled pressure reducing valve, without the need of a blower. In this case, the breathing valve should also be controllable.

In an actual prototype embodiment, the radial blower 15 is, for example, powered by a DC motor servo controller 21, which allows for a variable motor speed and hence a variable pressure. The motor speed (and pressure) is set by a microcontroller of a controller 26 or computer of the base station 10 via an I²C serial interface. It is, however, not mandatory to vary the pressure. The advantage of a variable pressure is increased patient safety, as the lowest possible pressure can be selected and applied (depending on the patient's breathing pattern and ventilatory mode).

The pressure delivered by the blower 15 is measured with a pressure transducer 23 and monitored by a microcontroller. This is not mandatory, as the pressure delivered by a radial blower 15 remains nearly constant (for a given motor speed) over a wide range of gas flow. However, a pressure measurement allows for a easier detection of any malfunction of the blower 15 and upstream (and downstream) pneumatic components. It is also optional to measure the gas flow with a pneumotachograph or flow meter (not shown in FIG. 3), e.g. to detect leaks or device failure.

The expiratory (=expiration) tubing 40 coming from the patient via a port 25 of the base station 10 is connected to a negative pressure source 19. This is optional but allows for a compensation of the flow resistance of the expiratory tubing (including the endotracheal tube) during expiration. If this compensation is not needed, or if a high PEEP value (positive end-expiratory pressure at the patient's airways) has been selected, the expiratory outlet of the demand-flow valve can open directly to atmosphere, without the need of a tubing between the demand-flow valve and the base station 10 (not shown here).

In the actual prototype embodiment, a radial blower 19 generates a negative pressure of up to −100 mbar below atmosphere, typically −20 mbar. Other negative pressure sources such as a Venturi valve or different types of pumps are also possible. The blower outlet then opens to atmosphere 17. Optionally, particularly in the case of a blower as a pressure source, silencers 18, 20 or mufflers can be used to reduce high-frequency acoustic noise.

As with the positive pressure source 15, the radial blower 19 of the negative pressure source can be optionally controlled by a DC motor servo controller 21 in connection with a microcontroller. The negative pressure can be optionally monitored with a pressure transducer 22. For safety reasons, the least negative pressure can be selected and applied (depending on the patient's breathing pattern and ventilatory mode). It is also optional to measure the gas flow with a pneumotachograph or flow meter (not shown in FIG. 3), e.g. to detect leaks or device failure.

The controller 26 or computer in the base station 10 is optional and is useful if a variable positive or negative pressure, and a monitoring of the delivered positive and negative pressure, is desired. The controller 26 can moreover monitor the motor speed for the radial blowers 15, 19, the motor temperature, or the temperature within the base station 10, for safety reasons. A RS232 serial connection may serve as an interface 29 to and from the demand-flow valve (as explained later) and may be looped through the microcontroller of the controller 26 so that the base station 10 can be (optionally) informed about the currently needed pressure and demand flow. Also optionally, the controller or computer provides a user interface 27, 28 and communicates with a controller 90 of the demand-flow valve 81 (see FIG. 8) e.g. to set ventilatory parameters or control parameters.

A computer or controller with a display 28 or screen and an input device 27 (e.g. mouse, touch screen, keyboard), a notebook, a tablet computer or similar device can be used as a user interface 27, 28 to
Display pressure, flow, volume diagrams, ventilator parameters (e.g. respiratory rate, tidal volume, minute ventilation)
Display and change the current settings of the ventilator 1 (e.g. ventilatory mode, PEEP, pressure support level, tube compensation, proportionality factors for proportional assist ventilation, trigger thresholds, target minute ventilation, alarms)
Display, change or calibrate the control parameters for the demand-flow controller 90 (e.g. PID parameters, pressure and flow calibration, zero position for the rotary encoder)
Store and retrieve measurements and log files
Communicate with, service, or re-program the demand-flow and base station controllers 26, 90

The user interface software can be provided by a dedicated computer or controller, or it can be provided by either the demand-flow or base station controllers 26, 90. The interface may also include an interface to a hospital IT network, such as M-HIS/M-PDMS, or another network to exchange any data.

Next, the structure and functions of the valve-sensor-assembly 80, 80′, and in particular of the demand-flow valve 81, 81′, are explained in more detail using FIGS. 4 to 8. Thereby, the FIGS. 4, 5, 6 and 8 show a first prototype embodiment of a valve-sensor-assembly 80. which is also shown in FIG. 2, and FIG. 7 shows a second prototype embodiment of a valve-sensor-assembly 80′. The first embodiment comprises a demand-flow valve 81, which has a gas flow from an inlet to an outlet in-plane, and the second embodiment comprises a demand-flow valve 81′, which has a gas flow outlet perpendicular to the inlets, as described later.

In the following, the first embodiment will be described first, whereby the explanations regarding the first embodiment, in particular regarding the mode of operation, apply in the same way to the second embodiment if not explicitly mentioned otherwise.

FIGS. 4 and 5 show the first prototype embodiment of the valve-sensor-assembly 80 from two different views from the outside. This valve-sensor-assembly 80 comprises a Y-piece 8Y that connects the two tubes 30, 40 for the inspiratory and expiratory flow circuit to the patient. For example, at the tube 30 for the inspiratory flow circuit an air/oxygen mixture with a positive pressure, e.g. +40 mbar, and at the tube 40 for the expiratory flow circuit a negative pressure, e.g. −20 mbar, (or atmospheric pressure) may be provided.

As can be seen from these figures, the Y-piece 8Y housing the demand-flow valve 81, connects—in direction to the patient—to a pneumotachograph or other device for flow sensing (flow sensor) 86, which connects to a bacterial filter/humidifier 51 followed by the short flexible tube 50 that connects to the swivel connector (see FIG. 2) and the endotracheal tube or other patient interface.

A controller 90 with the pressure sensors is in a housing on an upper side of the Y-piece 8Y. Via short tubes 86l that lead from the flow sensor (here a pneumotachograph) 86 to the pressure sensors, the pressure difference across a resistive element in the flow sensor 86 may be determined, as explained later in connection with FIG. 8.

A principal view from the inside of the Y-piece 8Y with the demand-flow valve 81 is shown in FIG. 2 and also in FIG. 6.

As can be seen in FIGS. 2 and 6, in the first prototype embodiment, the demand-flow valve 81 is a rotating 3-way valve which connects the outlet 85 (towards the patient) of the valve housing 82 either to the positive or negative side (i.e. with a connection tube or port 83 to the inspiration tubing 30 and a connection tube or port 84 to the expiration tubing 40). The rotation is facilitated by a servo motor 96 (FIG. 5), but a solenoid or rotating solenoid or other electric motor is also possible.

FIG. 6 shows in the middle row schematics of the demand-flow valve 81 from the left to the right in four different rotational positions of the rotatable closing element 89 (also referred to more briefly as “valve positions”), which may be rotated around a central axis A in the valve housing 82. The bottom row shows the diamond shape of the (valve) orifice 89o of the closure element 89 of the 3-way valve in the respective positions relative to the circular (inlet) orifice 83o of the connecting tube 83 of the valve housing 82 to the inspiratory tubing 30 and to the circular (inlet) orifice 84o of the connecting tube 84 to the expiratory tubing 40. The opening 89t of the closing element 89 in direction to the outlet 85 is dimensioned to fully overlap with the outlet 85 in any of the rotational positions shown in the middle row. In the upper row of FIG. 6, the airway pressure P_{aw} at the outlet 85 is shown as a function of the position of the closing element 89 shown in the middle row.

As can be seen from FIG. 6, a counter-clockwise rotation will increase the airway pressure P_{aw}, and a clockwise rotation will reduce the airway pressure P_{aw}. In the first position in FIG. 6 (and in FIG. 2; please note that FIG. 6 and FIG. 2 show the valve schematically from opposite sides) the demand-flow valve 81 is shown in the “inspiration” position. Here the airway pressure P_{aw} is the maximum pressure Pₘₐₓ (actually delivered by the pressure source, e.g. the blower 15 (see FIG. 3)). In the second position in FIG. 6, the pressure to the outlet 85 towards the patient is reduced by the demand-flow valve 81. The third position is the “neutral position” NP. In the neutral position NP, a (optional) small overlap with the positive and negative pressure orifice allows flow to pass between the positive and negative side to improve pressure control accuracy under low-flow conditions (e.g. end of expiration). In the fourth position, the demand-flow valve 81 is shown in the “expiration” position where the overlap of the orifice 89o of the closure element 89 with the orifice 84o of the connecting tube 84 to the expiratory tubing 40 is at its maximum. Here, the airway pressure P_{aw} is the maximum negative pressure (=minimal pressure) Pₘᵢₙ (actually delivered by the pressure source, e.g. the blower 19 (see FIG. 3)).

The gas flow is determined by the flow resistance and the pressure difference acting across the inlet orifice 83o, 84o—valve orifice 89o pathway. The flow resistance in turn is determined by the overlap area between the two orifices (83o, 89o or 84o, 89o in FIG. 6). By increasing the overlap, the internal gas flow resistance of the flow-demand valve 81 is reduced, and the resulting gas flow increases. The shape of the orifice 89o of the rotary closure element 89 and the shape of the orifices 83o, 84o of the pressure inlets 83, 84 determine the shape of the overlapping area and hence the valve resistance as a function of the rotational angle. As in the first prototype embodiment the inlet orifices 83o, 84o both have a circular shape, and the orifice 89o of the rotating closure element 89 has a diamond shape, the overlap area between the two orifices depends approximately quadratically on the angular valve position up to a relative area overlap of approximately 50%. For a constant pressure difference, this results in an approximately linear relationship between gas flow and angular valve position. For a larger overlap than 50%, the flow increases less-than-proportional with increasing angular position until a maximum flow is reached when the overlap reaches a maximum.

Since the orifice of the rotary valve can overlap with the orifice of only one of the two pressure inlets at any given time (in a typical configuration), the rotational valve position also determines the direction of the gas flow (inspiration or expiration), and hence the valve can be used to control the airway pressure both during inspiration and expiration.

In the neutral position NP, the orifice of the rotary valve typically does not overlap with any of the two orifices of the pressure inlets, effectively sealing or occluding both inlets and hence also the outlet so that no gas can flow. However, as explained above for the first embodiment, it may be desirable that in the neutral position, a small gas flow is allowed to by-pass from the positive to the negative inlet so that the pressure outlet is never fully occluded. Such a by-pass flow may improve the accuracy and speed of the valve when switching between inspiration and expiration and vice versa. A by-pass flow can be achieved by an orifice of the rotary valve that is slightly larger than the distance between the orifices of the two pressure inlets, as shown in FIG. 6.

In a preferred embodiment (not shown) the demand-flow valve comprises a mechanism to be adjusted between a first setting in which a by-pass flow is possible in the neutral position NP and a second setting in which both inlets are fully closed in the neutral position NP. Therefore, the closure element may be preferably mounted in the valve housing such that it is slightly displaceable relative to the orifices 83o, 84o of the pressure inlets 83, 84 along the axis of rotation A. With such a mechanism, depending on the axial position of the closure element, the overlap between the orifice 89o of the rotary closure element 89 and the orifices 83o, 84o of the pressure inlets 83, 84 in the neutral (rotational) position NP may be changed. Preferably, there is an overlap in the neutral position NP in a first axial position and there is no overlap in the neutral position NP in a second axial position, i.e. the valve is completely closed in the second axial position.

In particular, such a mechanism may be realized in an embodiment with a diamond-shaped orifice 89o of the rotary closure element 89 and circular orifices 83o, 84o of the pressure inlets 83, 84, as shown schematically in the bottom line in FIG. 6. Then, a slight displacement in axial direction is sufficient for the tips of the diamond-shaped orifice 89o) (as shown in the bottom row, in the 3rd picture from the left in FIG. 6) to no longer overlap with the circular orifices 83o, 84o.

This can also be realised particularly well with a design similar to the embodiment as shown in FIG. 7.

In the first embodiment, the pressure inlets connect to the valve housing in a straight arrangement and, as mentioned above, the outlet is in-plane with the inlet. In the embodiment according to FIG. 6, the pressure inlets 83′, 84′ (connecting tubes 83′, 84′ to the inspiration tubing 30 and the expiration tubing 40) connect to the valve housing 82′ in a curved arrangement and the outlet 85′ is perpendicular to the pressure inlets 83′, 84′. Curved pressure inlets in combination with a perpendicular outlet allow for a more compact design and may further reduce the inner volume of the valve (dead space). A grid structure or array of laminar tubes can be inserted into the valve outlet 85′ (not shown in FIG. 7) to prevent flow turbulence in a flow meter or pneumotachograph connected to the outlet 85′,

In the example embodiment shown in FIG. 7, the valve housing 82′ of the demand-flow valve 81′ (which in total also forms a kind of Y-Piece 8Y) is essentially made up of a central part 82a′ extending annularly around an axis of rotation A′, a lid 82b′ arranged on a first end face (front right in the figure) as well as a base part 82c′ on the other end face of the central part 82a′. In an assembled state, the central part 82a′ is sealed against the lid 82b′ and the base part 82c′ by ring seals 100, 101 respectively. The outlet 85′ is arranged coaxially to the axis of rotation A′ on the lid 82b′. Two pressure inlets 83′, 84′ are connected radially to the central part 82a′ and are curved to extend along the sides of the central part 82a′ in an opposite direction to the outlet 85′.

A closure element 89′ with a diamond-shaped valve orifice 89o′ is arranged inside the central part 82a′. This closing element 89′ is rotatable mounted between two ball bearings 102, 103.

On the side of the base part 82c′ facing away from the central part 82a′, the base part 82c′ is flanged to a motor 104 which is coupled to the closure element 89′ via a shaft (not shown) running along the axis of rotation A′ in order to rotate the closure element 89′ in a controlled manner. This motor 104 is arranged in a motor housing 105. On one side of the motor housing 105, a (relatively flat, C-shaped) housing 106 for the electronic components 108 (comprising motor drivers, valve controllers, pressure sensors, encoders etc.) is arranged. On the side facing away from the motor 104, the electronics housing 106 is covered by a panel 107 and on the front side near to the valve housing 82′ by a corner element 110, in which two ports 109 for the pressure tubes to the pressure sensors of the electronics to the flow sensor (not shown) are located. On the front side facing away from the valve housing 82′, the motor housing 105 and the electronics housing 106 have a common lid 111.

This design is particularly space-saving, for example, and can therefore be easily accommodated in the vicinity of the patient.

It will be understood that the electronic components of the electronics 108 are suitably connected (directly or indirectly) to the motor 104 and any further elements to be controlled and/or measuring elements, as will be explained later.

Regardless of the specific embodiment, the task of the demand-flow valve 81, 81′ according to the invention is to mix the pressure from the two pressure inlets (one being positive (e.g. typically +40 mbar), the other being negative (e.g. typically −20 mbar)) to a desired airway pressure. An alternative way of describing the function of demand-flow valve 81, 81′ is that it delivers as much gas flow from the two inlets as is needed to maintain a desired airway pressure or a desired gas flow to or from the patient. The direction of the gas flow from the positive pressure inlet is always towards the demand-flow valve 81, 81′, the direction of the gas flow from the negative pressure inlet is always away from the demand-flow valve 81, 81′. The direction of the gas flow at the outlet can be bi-directional. The patient's inspiration is defined as a positive flow away from the valve towards the patient; expiration is defined as a negative flow towards the demand-flow valve away from the patient. Unlike the demand-flow control method and valve used in traditional ventilators, which is active only during inspiration while a separate expiratory valve controls the pressure and flow during expiration, the demand-flow valve described here controls the airway pressure (or gas flow) both, during inspiration and expiration.

The following explanations regarding the controller design of the valves and regarding the devices and methods for measuring and/or recording the values of pressure and gas flow are also applicable to any other embodiment of the demand-flow valve, in particular to the demand-flow valve 81′ of the second prototype embodiment, even if the explanations are mainly based in connection with the first embodiment example.

The rotation of the demand-flow valve (respectively, of the closure element 89, 89′ of the demand-flow valve 81, 81′) can be achieved by a motor (as said above) or other form of actuator. In the second prototype, a stepper motor and integrated stepper motor driver are used, but any other actuator, e.g. a DC-motor, servomotor (as in the first prototype) or pneumatic actuator are also possible. The rotational position of the demand-flow valve (respectively, of the closure element of the demand-flow valve) may be measured with a magnetic encoder. The actuator (e.g. motor) and the valve (with the closure element inside the valve housing) are preferably coupled via a plug-in connection so that the parts of the valve unit that are in direct contact with oxygen/air to and from the patient can be separated from the actuator and controller for cleaning and sterilization or for an exchange, if desired.

For a demand-flow control of the demand-flow valve 81, 81′ as used for mechanical ventilation of patients, the controlled variable is the airway pressure, which should preferably be accurately and continuously measured without noticeable delays. In some modes of mechanical ventilation, e.g. for Automatic Tube Compensation (ATC) with or without Proportional Assist Ventilation (PAV), the target airway pressure is further modulated by the currently acting gas flow, which therefore should preferably also be accurately measured without delays.

Gas flow can be measured by various means, e.g. by heat wire anemometry or vortex detection. In the prototype embodiments, for example, the gas flow is measured from the pressure difference across a small resistive element (pneumotachograph). The pneumotachograph 86 is located between the demand-flow valve 81, 81′ and the patient's airways. One of the two pressure lines of the pneumotachograph 86 (typically the pressure line at the patient-side) can be split for measuring the airway pressure using a separate pressure sensor, as depicted in FIG. 2 and FIG. 8.

As mentioned above, flow and pressure measurements are subject to delays caused e.g. by the speed of sound or the low-pass characteristics from the compliance of the tubing. Furthermore, rapid pressure changes excite oscillations of the air column in the tubing, leading to substantial measurement errors. To reduce errors and avoid measurement delays, the distance between the measuring point and the pressure sensors and hence the length of the tubing should preferably be as short as possible. In the prototype, this distance is below 10 cm. Miniature high accuracy silicon ceramic piezoresistive pressure transducers (Honeywell HSC series) may be used both for measuring airway pressure and gas flow.

The electronic components that are part of the valve-sensor-assembly 80 (demand-flow valve and sensor unit) are depicted in FIG. 8. In this figure, dashed lines depict pneumatic components.

As can be seen here, the pressure sensors for detecting the pressures (and gas flow) in the pneumotachograph 86, which may be connected to different parts of the pneumotachograph 86 via small and short pressure tubes, may be also part of the controller 90 (as in the first and second embodiment) or at least arranged in the controller housing.

Two important electrical components are the differential pressure transducers (pressure sensors 97, 98) for measuring the gas flow and the airway pressure, to be placed as close as possible to the patient's airway opening, as explained in the previous section. All other components depicted in FIG. 8 except for the rotational encoder 93 for measuring the valve position (a motor driver 92 for powering the valve actuator/motor; a μ-controller 91 for carrying out the demand-flow control algorithm (controlling the motor and valve position, computing the resistive losses across the endotracheal tube etc. depending on the mode of ventilation, communication with the base station)) could be placed further away, e.g. at the base station 10 (see FIG. 2). However, that would require are substantial number of electrical signal and power lines between the base station 10 and the valve-sensor-assembly 80, which may compromise the quality of the electrical signals. By placing all electrical components of the valve-sensor-assembly 80 in a compact housing, as depicted in FIGS. 7 and 8, the only electrical connections between the base station 10 and the valve-sensor-assembly 80 are power supply (GND, +24 V) lines 94 and serial RS232 signal (GND, Rx, Tx) lines 95.

In the prototype, the motor driver 92 is a Trinamic TMC2209, the magnetic rotational encoder 93 is an AMS AS5600, and the μ-controller 91 is a PJRC Teensy 4.0 based on the ARM Cortex-M7 600 MHz processor. The μ-controller 91 may send a brief digital pulse to the motor driver 92 for every micro-step of the stepper motor (not shown in FIG. 8). In addition, the μ-controller 91 may set the direction and micro-step size via digital (TTL) signals. The magnetic rotational encoder 93 may send the current position of the stepper motor and hence the valve position to the μ-controller 91 via an I²C serial communication line, to ensure that no steps have been missed.

The μ-controller 91 may read the current airway pressure and differential pressure across the pneumotachograph 86 via I²C serial communication lines, compute the gas flow and delivered volume, compute the pressure drop across the endotracheal tube, compute the target airway pressure depending on the mode of ventilation, and control the position of the demand flow valve according to a feedback control algorithm (in the prototype, preferably a PID controller was chosen). The μ-controller 91 sends via an RS232 communication line the airway pressure and flow signals to the base station 10 and receives commands and settings from the base station 10.

If the currently measured airway pressure is lower than the target airway pressure, the PID controller tends to rotate the demand-flow valve 81, 81′ (respectively, the orifice 89o, 89o′ of the closure element 89, 89′ of the demand-flow valve 81, 81′) more towards the positive pressure inlet. Conversely, if the currently measured airway pressure is larger than the target airway pressure, the PID controller tends to rotate the demand-flow valve more towards the negative pressure inlet. It is also possible to directly control not the pressure but the gas flow, e.g. to emulate a volume-controlled mode of ventilation with a constant inspiratory flow rate. In this case, if the currently measured gas flow is lower than the target flow, the PID controller tends to rotate the demand flow valve more towards the positive pressure inlet, and vice versa. The parameters of the PID controller are optimized depending on the performance of the actuator/motor and the valve resistance to achieve maximum speed and accuracy with a minimum of pressure oscillations. In the second prototype, the PID controller is combined with a stepper motor controller that ensures that the acceleration of the motor remains within its physical limits.

As mentioned above, the target airway pressure may be set according to any of the established ventilatory modes, but preferably a ventilatory mode like ATC with or without PAV is used. In these modes, the target pressure P_{target} can be determined according to Equation (1) above and the pressure drops ΔP(ETT), ΔP(R_{aw}), ΔP(Eᵣₛ) can be computed based on the continuously measured gas flow (V′) and volume (V) according to Equations (2-6c) with the explanations above.

The performance of the demand-flow valve and method according to the invention compared to the performance of a commonly used leading commercial state-of-the-art intensive care ventilators (as comparison products) has been tested.

Thereby, two ventilatory modes were tested:
1) CPAP (Continuous Positive Airway Pressure), and
2) ATC (Automatic Tube Compensation)
   for a 7 mm diameter endotracheal tube (Rüsch, 32 cm long, flow resistance including swivel connector and flexible tubing at 1 l/s is ˜12 mbar s/l).

In CPAP mode, the ventilator is supposed to control the airway pressure P_{aw} to a constant value, regardless of the inspiratory or expiratory flow generated by the patient. In ATC mode, the ventilator is supposed to control the tracheal pressure P_{trach} to a constant value, regardless of the inspiratory or expiratory flow generated by the patient.

In the following, the test result for one comparison product (Hamilton S1) are presented. The test set-up is schematically shown in FIG. 9.

Either the ventilator 1 (the second prototype embodiment of the valve-sensor-assembly 80′), or the comparison product (conventional ventilator 2) are connected to the ventilator unit side of a pneumotachograph 86, which is on the patient-end connected to a bacterial filter 210, further to a flexible tube 50 and via a swivel connector 60 to an ETT 70. Note, that in FIG. 9 a symbolic representation similar to the first embodiment is shown, as this symbolism is more self-explanatory.

The flow is generated by a spontaneously breathing test subject (not shown).

The airway pressure P_{aw} is measured at the patient-end of the pneumotachograph 86; the tracheal pressure P_{trach} is measured 5 cm behind the patient-end of the ETT 70 that is intubated and firmly attached to an artificial tracheal 211 (a tube with an inner diameter of 2 cm) by inflating the tube cuff. The other end of the artificial trachea 211 is connected to a mouthpiece 212 for the test subject.

Note that the pneumotachograph 86 introduces a small additional flow resistance of ˜0.7 mbar s/l, which the commercial ventilator does not “know” about and therefore does not compensate for in CPAP or ATC mode. Also, for hygienic reasons, the bacterial filter 210 was added between the ventilator and the test subject. This bacterial filter 210 introduces an additional flow resistance of ˜2 mbar s/l that acts in series with the endotracheal tube resistance. This is of no consequence for comparing the ventilators in CPAP mode but makes a difference in ATC mode. Although such bacterial filters are commonly used in ventilated patients, commercial ventilators do not provide an option to compensate for its flow resistance. The prototype ventilator 1, by contrast, was programmed to also compensate for the resistance of the bacterial filter.

In FIGS. 10 and 11, the measurement diagrams with the comparison product are shown on the left, and the corresponding measurement diagrams with the prototype according to the invention are shown on the right for better comparison.

FIG. 10 shows the airway pressure P_{aw} (top diagrams, in mbar) and the flow fl (bottom diagrams; in l/s) over time t (in s) during 3 consecutive spontaneous breaths under CPAP mode (continuous positive airway pressure). The positive end-expiratory pressure (PEEP) was set to 0 mbar. Therefore, the airway pressure P_{aw} is expected to remain constant at 0 mbar. Deviations from this target pressure are indicated by the hatched area.

The comparison product shows substantial deviations of the airway pressure P_{aw} from the target of about −2 . . . 3 mbar throughout inspiration, and about +4 mbar throughout expiration.

The prototype shows almost no deviations of the airway pressure P_{aw} from the target pressure. Only during switching from inspiration to expiration and vice versa does the airway pressure deviate briefly (˜100 ms) by up to 2 mbar from the target pressure.

The CPAP mode is routinely used to test for extubation readiness. If a patient is able to breathe under CPAP mode for 30 min without exhaustion, the patient is considered ready for extubation. The substantial deviations of the airway pressure P_{aw} from the target pressure, however, impose a large additional work of breathing during inspiration and impede expiration. This may cause a patient who is ready for extubation to erroneously be considered as not-ready. By contrast, the prototype is able to control the airway pressure precisely to the target pressure, except for a short (˜100 ms) period during switching between inspiration and expiration where measurable deviations can occur.

A similar test with 3 other commercial ventilators under CPAP was recently published (Sameed, Chatburn et al. 2023), and these published data also show large deviations of the airway pressure from the target pressure during inspiration and expiration, prompting the recommendation of the authors that CPAP should no longer be used to test for extubation readiness in patients. Taken together, these measurements demonstrate the superior performance of the prototype ventilator compared to state-of-the-art commercial ventilators.

FIG. 11 shows the airway pressure P_{aw} (dotted line), the tracheal pressure P_{trach} (solid line) (top diagrams, in mbar), and the flow fl (bottom diagrams, in l/s) over time t (in s) during 3 consecutive spontaneous breaths under ATC mode (automatic tube compensation). The positive end-expiratory pressure (PEEP) was set to 0 mbar. Therefore, the tracheal pressure P_{trach} is expected to remain constant at 0 mbar. Deviations from this target pressure are indicated by the hatched area.

The comparison product does not increase the airway pressure above PEEP during inspiration, and hence the tracheal pressure P_{trach} remains far below target pressure. During expiration, the airway pressure P_{aw} fails to decrease below PEEP, and the tracheal pressure remains far above the target pressure.

In the prototype, the airway pressure P_{aw} increases far above PEEP during inspiration, and falls far below PEEP during expiration. The tracheal pressure P_{trach} remains close to the target pressure. Only during switching from inspiration to expiration and vice versa does the tracheal pressure P_{trach} deviate briefly (˜100 ms) by up to 5 mbar from the target pressure. Note that the test subject generated a larger inspiratory and expiratory flow (compare to breathing with a commercial ventilator) due to the complete tube compensation provided by the prototype, which prevents flow-limiting additional work of breathing.

Taken together, the comparison product does not provide any tube resistance compensation whatsoever, neither during inspiration nor expiration. By contrast, the prototype always compensates nearly fully for the tube resistance, demonstrating its superior performance compared to a leading state-of-the-art commercial ventilator.

Therefore, as explained above, in a preferred embodiment, the invention combines a
Sub-critical low pressure demand flow vale in close proximity to the patient
Pressure and flow sensors in close proximity to the patient
Safe inspiratory circuit (approximately +40 mbar) for driving inspiratory support
Safe expiratory circuit (approximately −20 mbar) for driving expiratory support
ATC, flow and volume-proportional assist mode
   optimized for the support of spontaneously breathing intensive care patients, in particular patients with ARDS.

Therefore, the invention allows a design of ventilation support to minimize the additional work of breathing caused by the resistance of the endotracheal tube, the resistance of the humidifier, the resistance of the expiratory tubing and expiratory valve, and the delay of the demand flow and the feedback control signals (airway pressure gas flow) caused by the finite speed of sound in elastic tubes. In addition, the invention avoids patient-ventilator desynchronization by using the ATC mode in combination with flow- and volume assist if required, thereby controlling the tracheal pressure (or alveolar, pleural or muscle pressure) instead of delivering a pre-defined pressure support.

This will allow ARDS patients and other patients with respiratory insufficiency to breath spontaneously. The invention may therefore benefit in particular those patients that currently cannot be supported with state-of-the-art pressure support modes that cause additional work of breathing and patient-ventilator desynchronization. The invention may assist each inspiratory and expiratory effort of the patient with a level of pressure support (or pressure release during expiration) that is synchronized to the patient's effort, with minimal delay, and a minimum of intrathoracic pressure above PEEP. The invention may therefore allow intensive care physicians to employ a strategy of ventilator support that is expected to reduce ventilator-induced baro- and volutrauma to the lungs, and therefore to a decrease the mortality of ventilated ARDS patients.

Although the present invention has been disclosed in the form of preferred embodiments and variations thereon, it will be understood that numerous additional modifications and variations could be made thereto without departing from the scope of the invention. In particular, unless otherwise explicitly mentioned, individual features or feature groups of an embodiment may also usefully develop the invention independently of other elements of that specific embodiment. For example, the type of motor/actuator used for the first embodiment may also be used for the second embodiment or vice versa. For the sake of clarity, it is to be understood that the use of “a” or “an” throughout this application does not exclude a plurality, and “comprising” does not exclude other steps or elements. The mention of a “unit” or a “device” does not preclude the use of more than one unit or device. The expression “a number of” has to be understood as “at least one”.

### LIST OF REFERENCES

Fabry, B., J. Guttmann, L. Eberhard and G. Wolff (1994 a). “Automatic compensation of endotracheal tube resistance in spontaneously breathing patients.” Technol Health Care 1(4): 281-291.
Fabry, B., J. Guttmann, L. Eberhard and G. Wolff (1994 b). “Treatment of patient-ventilator desynchronization with automatic tube compensation (ATC) and proportional assist ventilation.” Int Care Med 20(Suppl. 2): 38.
Fabry, B., J. Guttmann, L. Eberhard, T. Bauer, C. Haberthur and G. Wolff (1995). “An analysis of desynchronization between the spontaneously breathing patient and ventilator during inspiratory pressure support.” Chest 107(5): 1387-1394.
Fabry, B., C. Haberthur, D. Zappe, J. Guttmann, R. Kuhlen and R. Stocker (1997). “Breathing pattern and additional work of breathing in spontaneously breathing patients with different ventilatory demands during inspiratory pressure support and automatic tube compensation.” Intensive Care Med 23(5): 545-552.
Guttmann, J., L. Eberhard, B. Fabry, W. Bertschmann and G. Wolff (1993). “Continuous calculation of intratracheal pressure in tracheally intubated patients.” Anesthesiology 79(3): 503-513.
Guttmann, J., L. Eberhard, B. Fabry, W. Bertschmann, J. Zeravik, M. Adolph, J. Eckart and G. Wolff (1995). “Time constant/volume relationship of passive expiration in mechanically ventilated ARDS patients.” Eur Respir J 8(1): 114-120.
Sameed, M., R. L. Chatburn and U. Hatipoglu (2023). “Bench Assessment of Work of Breathing During a Spontaneous Breathing Trial on Zero Pressure Support and Zero PEEP Compared to T-Piece.” Respir Care 68(6): 767-772.

### REFERENCE SIGNS LIST

1 ventilator/ventilator device
2 state-of-the-art ventilator
3 demand-flow valve
4 sensors
5 sensors
6 outlet valve
7 ventilation unit
8,8Y Y-piece
10 base station
10h housing
11 air-oxygen blender
11a, 11b ports
11c, 11d pressure reducing valves
12 pressure regulator/pressure reducing valve
13 breathing valve (pressure/vacuum relief valve)
14 port
15 pressure source/blower
16 silencer
17 outlet/atmosphere
18 silencer
19 pressure source/blower
20 silencer
21 servo controller
22, 23 pressure transducer
24, 25 port
26 controller
27 user interface/input device
28 user interface/display
29 interface
30 first tube/inspiration tube/inspiration tubing
40 second tube/expiration tube/expiration tubing
50 flexible tube
51 bacterial filter/humidifier
60 swivel connector
70 patient interface/endotracheal tube (ETT)
80, 80′ valve-sensor-assembly
81,81′ demand-flow valve
82, 82′ valve housing
82a′ central part
82b′ lid
82c′ base part
83, 83′, 84, 84′ connecting tubes/pressure inlets/ports
83o, 84o inlet orifice
85, 85′ outlet
86 measuring device/flow sensor/pneumotachograph
86l tubes
87,88 measuring devices/pressure sensors
89, 89′ closure element/shutter
89o, 89o′ valve orifice
89t opening
90 controller
91 μ-controller
92 motor driver
93 rotational encoder
94 power supply lines
95 signal lines
96 servo motor
97, 98 differential pressure transducers/pressure sensors
100, 101 ring seals
102, 103 ball bearings
104 motor
105 motor housing
106 electronics housing
107 panel
108 electronics
109 port
110 corner element
111 common lid
210 bacterial filter
211 artificial trachea
212 mouthpiece
a Distance
A, A′ axis
fl flow
NP neutral position
P_{aw} airway pressure
P_{trach} tracheal pressure
Pₘₐₓ maximal pressure
Pₘᵢₙ minimal pressure/maximal negative pressure
t time

1. Device (1) for providing ventilatory assistance to a patient, comprising
a set of tubes (30, 40, 50, 60, 70) for gas flow (fl) to and from the patient,
a demand-flow valve (81, 81′),
a flow sensor (86),
and
a pressure sensor (87, 88) for measuring the airway pressure,
wherein the demand-flow valve (81, 81′) is located at a Y-piece (8Y) that connects a tube (30) for the inspiratory and a tube (40) for the expiratory flow circuit to the patient.

2. Device according to claim 1, wherein the demand-flow valve (81, 81′) is realized and positioned such that a maximum dead space in the demand-flow valve (81) and in a tube system (86, 50, 60, 70) from the demand-flow valve (81, 81′) to a coupling point of a patient interface is 100 cm³, preferably 60 cm³, more preferably 55 cm³, further more preferably 30 cm³.

3. Device according to claim 1, wherein the two tubes (30, 40) for the inspiratory and expiratory flow circuit that connect to the Y-piece (8Y) are connected to a pressure source (15, 19) delivering a positive pressure, preferably around +40 mbar, for the inspiratory circuit, and a negative pressure, preferably-20 mbar, for the expiratory circuit, wherein other pressure levels can be chosen if needed, including atmospheric pressure.

4. Device according to claim 1, wherein the Y-piece (8Y) houses the demand-flow valve (81, 81′).

5. Device according to claim 1, wherein the demand-flow valve (81) is a 3-way valve (81, 81′).

6. Device according to claim 1, wherein the flow sensor (86) and the pressure sensor (87, 88) are placed in close proximity to the patient, preferably on the patient's side of the demand-flow valve (81, 81′).

7. Device according to claim 1, wherein the Y-piece (8Y) connects to a pneumotachograph (86) or other device for flow sensing, which optionally connects to a bacterial filter and/or humidifier (51), which is preferably followed by a short flexible tube (50) that connects to a swivel connector (60) and an patient interface (70).

8. Device according to claim 1, comprising a means (26, 90, 91) of controlling the demand-flow valve (81, 81′), and preferably also of controlling the pressure sources (15, 19), depending on a predefined mode of ventilator support.

9. Device according to claim 1, wherein the demand-flow valve (81) is realized such that in a neutral position of the demand-flow valve (81), a small overlap allows flow to pass between the positive and negative side.

10. Device according to claim 1, comprising at least
a valve-sensor-assembly (80, 80′), comprising the demand-flow valve (81, 81′) and the sensors (86, 87, 88) and
a base station (10), which is located remotely from the valve-sensor-assembly (80, 80′), comprising means (15, 19) for the positive and negative pressure.

11. Device according to claim 1, wherein the set of tubes (30, 40, 50, 60, 70) for gas flow comprises at least one endotracheal tube (70) or tracheostomy tube which
has, in an adult version, a maximum inner diameter of 7 mm, preferably 6 mm, more preferably 5 mm, and if feasible even smaller,
or
has, in a child or infant version, a maximum inner diameter of 5 mm, preferably 2 mm.

12. A demand-flow valve (81, 81′), in particular a valve-sensor-assembly (80, 80′), for a device (1) according to claim 1.

13. Method for controlling a ventilator (1), comprising the steps of:
measuring a gas flow with a flow sensor (86),
measuring an airway pressure with a pressure sensor (87, 88),
controlling a demand-flow valve (81, 81′) based on the gas flow and the airway opening pressure
whereby the demand-flow valve is located at a Y-piece (8Y) that connects a tube (30) for the inspiratory and a tube (40) for the expiratory flow circuit to the patient.

14. Method according to claim 13, wherein at least one of an airway pressure, a tracheal pressure, an alveola pressure, a pleura pressure or a muscle pressure is controlled by controlling the demand-flow valve (81, 81′).

15. Method according to claim 14, wherein the tracheal pressure, alveola pressure, pleura pressure or muscle pressure is calculated based on the gas flow and the airway opening pressure (Paw) and the gas flow (fl) is controlled via the demand-flow valve (81, 81′) to keep the tracheal pressure constant and/or to compensate fully or partially for the work of breathing required to overcome the airway resistance, lung elastance, and chest wall elastance.

16. Device according to claim 4, wherein the Y-piece (8Y) connects to a pneumotachograph (86) or other device for flow sensing, which optionally connects to a bacterial filter and/or humidifier (51), which is preferably followed by a short flexible tube (50) that connects to a swivel connector (60) and an patient interface (70).
